SEQUENCE LISTING (23 sequences)

SEQ ID NO: 1 - protein, 5 aa, Artificial Sequence
  Synthetic construct
DDDDK

SEQ ID NO: 2 - protein, 6 aa, Artificial Sequence
  Synthetic construct
LVPRGS

SEQ ID NO: 3 - protein, 7 aa, Artificial Sequence
  sythetic contruct
ENLYFQG

SEQ ID NO: 4 - protein, 8 aa, Artificial Sequence
  Synthetic construct
LEVLFQGP

SEQ ID NO: 5 - protein, 7 aa, Artificial Sequence
  Synthetic construct
  VARIANT 7: Xaa=Ser or Gly
  VARIANT 2, 3, 5,: Xaa = Any Amino Acid
EXXYXQX

SEQ ID NO: 6 - DNA, 35 bp, Artificial Sequence
  synthetic primer
gtaagctttcggggacatgggaatgcaaaagtagc

SEQ ID NO: 7 - DNA, 31 bp, Artificial Sequence
  synthetic primer
gtggatccgggctcaaaccatggagacccct

SEQ ID NO: 8 - DNA, 39 bp, Artificial Sequence
  sythetic primer
aagctttcggggacatggaaatgcaaaagagcggccttc

SEQ ID NO: 9 - DNA, 49 bp, Artificial Sequence
  sythetic primer
cagtgcccagggatgaaaacctgtattttcagggctgtggttgtaagcc

SEQ ID NO: 10 - protein, 19 aa, Artificial Sequence
  sythetic construct
PRDENLYFQGCGCKPCICT

SEQ ID NO: 11 - DNA, 29 bp, Artificial Sequence
  sythetic primer
ttataagcttcttaccttatgtgcttgtg

SEQ ID NO: 12 - DNA, 31 bp, Artificial Sequence
  sythetic primer
atatggatcctttgtctctaacactcattcc

SEQ ID NO: 13 - DNA, 21 bp, Artificial Sequence
  sythetic primer
tgctgtctttgctgtcctgat

SEQ ID NO: 14 - DNA, 21 bp, Artificial Sequence
  sythetic primer
tagaggtcagactgcaggaca

SEQ ID NO: 15 - DNA, 21 bp, Artificial Sequence
  sythetic primer
caagaagcacacgactgaggc

SEQ ID NO: 16 - DNA, 23 bp, Artificial Sequence
  sythetic primer
cttgaccaggcatcctagagtca

SEQ ID NO: 17 - DNA, 37 bp, Artificial Sequence
  sythetic primer
attctgcattccaaattgttctcacccagtctccagc

SEQ ID NO: 18 - DNA, 41 bp, Artificial Sequence
  sythetic primer
tcgacgtacgttctactcacgtttcagctccagcttggtcc

SEQ ID NO: 19 - DNA, 27 bp, Artificial Sequence
  sythetic primer
tctgcattccaggtccagctgcagcag

SEQ ID NO: 20 - DNA, 39 bp, Artificial Sequence
  sythetic primer
gacgtacgttctactcacctgaggagacggtgactgacc

SEQ ID NO: 21 - DNA, 19 bp, Artificial Sequence
  sythetic primer
ccaagctagcttggtaccg

SEQ ID NO: 22 - DNA, 20 bp, Artificial Sequence
  sythetic primer
ggatcctttgtctctaacac

SEQ ID NO: 23 - DNA, 19 bp, Artificial Sequence
  sythetic primer
gaattcgcccttgtggatc

(19) United States
(12) Patent Application Publication
(10) Pub. No.: US 2020/0109197 A1
(43) Pub. Date: Apr. 9, 2020

(54) RECOMBINANT ANTIBODY COMPOSITIONS AND METHODS OF USE THEREOF

(71) Applicant: LIFE TECHNOLOGIES AS, Oslo (NO)

(72) Inventors: Karoline Schjetne, Lommedalen (NO); Tanja Aarvak, Oslo (NO); Lars Norderhaug, Nesodtangen (NO)

(21) Appl. No.: 16/560,642

(22) Filed: Sep. 4, 2019

Related U.S. Application Data
(62) Division of application No. 15/433,181, filed on Feb. 15, 2017, now abandoned.
Application No. 15/433,181 is a division of application No. 14/402,463, filed on Nov. 20, 2014, now abandoned.
(60) Provisional application No. 61/650,388, filed on May 22, 2012.

(51) Int. Cl. C07K 16/28; C07K 16/00; C12N 5/0783

(52) U.S. Cl. CPC C07K 16/2803; C07K 16/00; C07K 16/2809; C07K 2317/55; C12N 5/0636; C07K 2317/53; C07K 16/2896

(57) ABSTRACT

Provided are methods and composition useful in isolating or otherwise preparing a population of target cells. In part, it relates to recombinant antibodies comprising an exogenous proteolytic cleavage site and nucleic acid molecules encoding the antibodies.

### CROSS REFERENCE TO RELATED APPLICATIONS

This application is a division of U.S. patent application Ser. No. 15/433,181 filed on Feb. 15, 2017, which is a division of U.S. patent application Ser. No. 14/402,463 filed on Nov. 20, 2014 which is a 35 U.S.C. § 371(c) of International Application No. PCT/US2013/029450 filed Mar. 6, 2013, which claims the benefit under 35 U.S.C. 119(e) of U.S. Provisional Patent Application No. 61/650,388, filed May 22, 2012, which is hereby incorporated by reference.

### SEQUENCE LISTING

This application hereby incorporates by reference the material of the electronic Sequence Listing filed concurrently herewith. The material in the electronic Sequence Listing is submitted as a text (.txt) file entitled “LT00692US_SEQLIST.TXT” created on Feb. 25, 2013, and is herein incorporated by reference in its entirety.

### TECHNICAL FIELD

The present invention relates to recombinant antibodies and their use in methods for isolating or otherwise preparing a population of target cells. In particular embodiments, it relates to recombinant antibodies comprising an exogenous proteolytic cleavage site and nucleic acid molecules encoding the antibodies.

### BACKGROUND

Isolated cells or cell compositions enriched for particular cells have long been of great interest and utility to the research and clinical communities and to the pharmaceutical industry. Accordingly, different procedures and reagents have been developed to prepare such isolated or enriched cell compositions from a less pure or mixed cell population. Various cell attributes have be used to segregate a particular cell from a mixed cell population including the size, shape, motility, and surface molecules of the target cell.

Antibodies which specifically associate with target molecules on the surface of the cells of interest have been used to separate the desired (or target) cells from other cells of a mixed population which do not have the particular surface molecule. For example, antibodies specific for particular cell surface molecules have been used to label cells of interest and then to positively or negatively select the labeled cells to prepare a desired cell population.

Antibody-coated surfaces, such as beads or culture wells, have been used to capture specific cells and to separate the bound cells from the unbound cells in the population. However, many uses for the isolated cells require removal of the cells from the capture antibody/surface complex and this removal can result in cell damage or alteration that leaves the isolated cells unsuitable for or incompatible with particular uses or downstream applications.

For example, methods which use a cocktail of proteolytic and collagenolytic enzymes to remove cells from a capture antibody/bead complex can degrade many cell surface molecules and alter at least the surface characteristics of the isolated cells. In other methods, cells are released from a capture antibody/bead complex by separating the capture antibody from the bead but leaving the capture antibody bound to the target cell surface. While still other cell isolation methods simply do not remove the capture antibody/bead complex from the isolated cells.

There remains a need for alternative procedures and reagents for cell isolation that, for example, produce specific cell populations without carrying over isolation reagents or modifying the cells in ways which may be undesirable for uses and further applications of the cells.

All patents, patent applications, and publications cited herein are hereby incorporated herein by reference in their entirety

### SUMMARY

Provided herein, in part, are methods for isolating a target cell from a mixed population using a recombinant antibody which specifically binds the target cell and a protease which cleaves an exogenous proteolytic cleavage site in the antibody. Also provided is a recombinant antibody comprising an exogenous proteolytic cleavage site, as well as compositions comprising the antibody. Also provided is a nucleic acid molecule and expression vector encoding the antibody.

In one aspect, provided herein is a method for detaching a target cell attached to a recombinant antibody-coated solid surface, the method comprising contacting the target cell attached to the surface with a protease, or portion of a protease, specific for an exogenous proteolytic cleavage site in the antibody, thereby detaching the target cell from the surface.

In another aspect, provided herein is a method for isolating a target cell from a mixed cell population, the method comprising a) contacting a mixed cell population with an antibody attached to a solid surface, wherein the antibody specifically binds to the target cell in the population and wherein the antibody comprises an exogenous proteolytic cleavage site. In some embodiments, the method further comprises b) separating the target cells bound to the surface from cells not bound to the surface; optionally further comprising c) contacting the target cells bound to the surface with a protease, or portion of a protease, specific for the cleavage site to detach the target cells from the surface; and optionally further comprising d) collecting the target cells detached from the surface.

In another aspect, provided herein is a method for producing a population of activated target cells, the method comprising a) contacting a cell population comprising target cells with an agonistic antibody attached to a solid surface, wherein the antibody specifically binds to the target cells in the population and wherein the antibody comprises an exogenous proteolytic cleavage site; and b) incubating the cells and the antibody attached to the surface under conditions to allow activation of the target cells. In some embodiments, the method further comprises c) separating the target cells bound to the surface from cells not bound to the surface; optionally further comprising d) contacting the target cells bound to the surface with a protease, or portion of a protease, specific for the cleavage site to detach the target cells from the surface; and optionally further comprising e) collecting the activated target cells detached from the surface.

In another aspect, provided herein is a method for positively isolating an antibody-free population of target cells, the method comprising: a) contacting a mixed cell population with an antibody attached to a solid surface, wherein the antibody specifically binds to the target cell in the population and wherein the antibody comprises an exogenous protease cleavage site; b) separating the target cells bound to the surface from cells not bound to the surface; c) contacting the target cells bound to the surface with a protease, or portion of a protease, specific for the cleavage site to detach the target cells from the surface; and d) collecting the target cells detached from the antibody and the surface to form a population of target cells free of bound antibody.

In another aspect, provided herein is a method for producing a bead-free population of target cells, the method comprising a) contacting a mixed cell population with an antibody attached to beads, wherein the antibody specifically binds to the target cell in the population and wherein the antibody comprises an exogenous protease cleavage site; b) separating the target cells bound to the beads from cells not bound to the beads; c) contacting the target cells bound to the beads with a protease, or portion of a protease, specific for the cleavage site to detach the target cells from the beads; and d) collecting the target cells detached from the beads to form a bead-free population of target cells.

In another aspect, provided herein is a recombinant antibody comprising an exogenous proteolytic cleavage site in the hinge region.

In another aspect, provided herein is a composition comprising a cell, a recombinant antibody, and a solid surface, where the recombinant antibody is specifically bound to the cell and is also attached to the solid surface, and where the recombinant antibody comprises an exogenous proteolytic cleavage site.

In another aspect, provided herein is a nucleic acid molecule encoding an antibody heavy chain comprising an exogenous proteolytic cleavage site in the hinge region.

In some embodiments, the protease is not expressed by the target cell.

In some embodiments, the protease is a viral protease. In certain embodiments, the protease is selected from the group consisting of a tobacco etch virus (TEV) protease, a rhinovirus 3C protease, a TVMV protease, a plum pox virus protease, and a turnip mosaic virus protease.

In some embodiments, the protease is an endoprotease. In certain embodiments, the protease is selected from the group consisting of enteropeptidase, thrombin and Factor Xa.

In some embodiments of the provided methods and compositions, the exogenous proteolytic cleavage site is for a protease not expressed in the target cell.

In some embodiments of the provided methods and compositions, the exogenous proteolytic cleavage site is for a viral protease. In certain embodiments, the exogenous proteolytic cleavage site is for a protease selected from the group consisting of a tobacco etch virus (TEV) protease, a rhinovirus 3C protease, a TVMV protease, a plum pox virus protease, and a turnip mosaic virus protease.

In some embodiments of the provided methods and compositions, the exogenous proteolytic cleavage site is for an endoprotease. In certain embodiments, the exogenous proteolytic cleavage site is for a protease is selected from the group consisting of enteropeptidase, thrombin and Factor Xa.

In some embodiments of the provided methods and compositions, the exogenous proteolytic cleavage site is in the hinge region of the antibody. In some embodiments, the exogenous proteolytic cleavage site is in the upper hinge region of the antibody.

In some embodiments, the target cell is a T cell, a stem cell, or a CD34+ cell.

In some embodiments, the recombinant antibody specifically binds to an endogenous protein on the target cell surface. In some embodiments, the recombinant antibody specifically binds to an exogenous protein on the target cell surface. In some embodiments, the recombinant antibody specifically binds a CD3 epitope, a Tra-1-60 epitope, or a CD34 epitope.

In some embodiments, the recombinant antibody is directly attached to a solid surface. In other embodiments, the recombinant antibody is indirectly attached to a solid surface. In some embodiments, the solid surface is a culture well or bead. In certain embodiments, the solid surface is a paramagnetic bead.

### BRIEF DESCRIPTION OF THE DRAWINGS

FIG. 1 A-B depict maps of high expression backbone H-chain (FIG. 1A, mIgG1-TEV SPV) and L-chain (FIG. 1B, mCK-SPV) vectors with V-regions encoding anti-human CD3 and genomic sequence of H- and L-chain genes. Restriction enzyme sites used for V-region or C-region switch are indicated.

FIG. 2 shows electrophoresis of recombinant antibodies (rAb TEV) under non-reduced (left) and reduced (right) conditions along with controls: molecular weight markers (MwM), purified mouse IgG1 (Std IgG1), spent supernatant from mock transfected cells (Neg), and control plasmids expressing human IgG (hIgG).

FIG. 3 A-B depict flow cytometry analysis of mononuclear cell (MNC) staining by anti-CD3-APC with (FIG. 3A) and without (FIG. 3B) prior treatment of the cells with rAb-TEV.

FIG. 4 shows electrophoresis of rAb TEV following incubation with AcTEV™ Protease (lanes 3 and 4) along with controls: molecular weight markers and rAb-TEV under non-reduced conditions (lane 1), rAB-TEV under reduced conditions (lane 2), control IgG with AcTEV™ Protease (lane 5). AcTEV™ Protease is 27 kDa in molecular weight.

FIG. 5 depicts flow cytometry analysis of rAb-TEV coupled to Dynabeads and detected by anti-mouse IgG1-FITC (top panel) along with controls: rAb-TEV coupled to Dynabeads+anti-mouse IgG2a-FITC (bottom left panel) and mock transfected supernantant coupled to Dynabeads+anti-mouse IgG1-FITC (bottom right panel).

FIG. 6 depicts flow cytometry histograms of rAb-TEV coupled to Dynabeads and detected by anti-mouse kappa-PE antibody before (top left) and after (bottom left and right) incubation with AcTEV™ Protease. A cartoon depicting the two scenarios is shown in top right. Incubation with AcTEV™ Protease was at room temperature (bottom left) or at 37° C. (bottom right). The percentage of kappa-positive beads is indicated in each histogram.

FIG. 7 is a chart depicting the percentage of depletion of CD3+ T cell from a MNC population using varying amounts of rAb-TEV bound per 10e7 beads.

FIG. 8 is a cartoon depicting an exemplary workflow for positive isolation of bead-free CD3+ T cells using rAb-TEV coupled to magnetic beads and TEV protease for bead release.

FIG. 9 depicts the recovery, purity and viability of isolated CD3+ cells using beads with varying amounts of attached antibody and the control performed without protease.

FIG. 10 is a chart showing the average recovery and purity of positively isolated CD3+ T cells from MNCs of healthy donors (n=7).

FIG. 11 A-C depict results showing release of capture antibody and availability of target epitope on cells following protease treatment.

FIG. 12 depicts results of activation and expansion of positively isolated CD3+ T cells: (FIG. 12A) fold cell expansion at day 6; (FIG. 12B) CD62L and CD27 expression on CD4 and CD8 cells.

FIG. 13 is a cartoon depicting exemplary compositions and use thereof for cell isolation. The scissors represents a protease specific for the exogenous proteolytic cleavage site in the antibody specific for the target cell epitope.

FIG. 14 A-C depict flow cytometry analysis of a mix of KG1a and FreeStyle™ 293F cells stained by anti-CD34-FITC: (FIG. 14A) without prior treatment of the cells with rAb-TEV anti-CD34; (FIG. 14B) with prior treatment of the cells with rAb-TEV anti-CD34; (FIG. 14C) with prior treatment of the cells with rAb-TEV anti-CD34 and AcTEV protease showing release of rAb-TEV anti-CD34 and availability of target epitope on cells following protease treatment.

FIG. 15 shows electrophoresis of rAb TEV anti-CD34 following incubation with AcTEV™ Protease (lanes 3 and 4) along with controls: molecular weight markers (lane 1), rAB-TEV anti-CD34 without protease (lane 2).

FIG. 16 depicts the recovery, depletion, and purity of isolated CD34+ cells using rAb-TEV anti-CD34 conjugated to beads with three different post-bead release wash conditions. The data shown are the average of triplicates.

### DETAILED DESCRIPTION

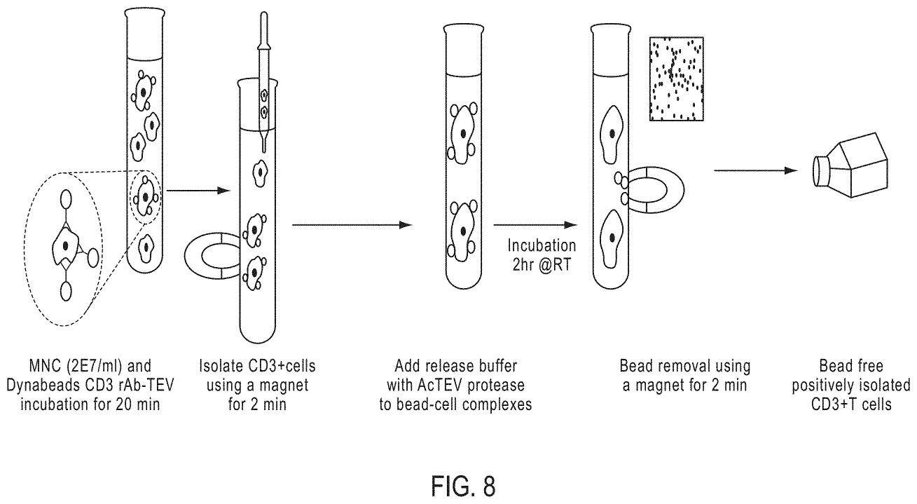

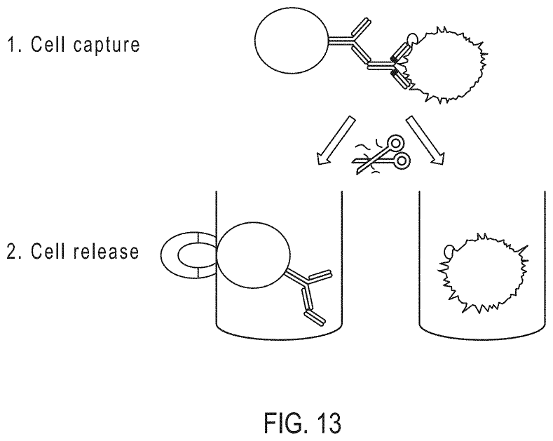

Disclosed herein are methods and compositions which relate, in part, to isolation of a population of target cells. The methods and compositions provided herein can be used to isolate a target cell population without the isolated cells carrying the isolation reagents which may induce or prevent signaling events when the cells are used in further applications. In exemplary embodiments, use of the provided methods and compositions permit the preparation of cells free of the biologically active portion of the capture antibody (the Fc portion) and free of the capture beads enabling the use of such a cell preparation for clinical and in vivo applications. See the illustrations in FIGS. 13 and 8 for a summary of several steps of the methods of particular embodiments and for a summary of an exemplary workflow, respectively.

The methods involve the use of an antibody which contains an exogenous proteolytic cleavage site, that is, an antibody which has been modified to include a proteolytic cleavage site that is not naturally found in the antibody. The methods also involve the use a protease with proteolytic activity specific for the exogenous cleavage site inserted in the antibody. The proteolytic cleavage site in the antibody can include any site at which cleavage occurs. Generally, as is known in the art, in addition to the amino acids at which the cleavage occurs, a proteolytic cleavage site also includes additional amino acid residues that help to define the protease specificity for the cleavage site.

The recombinant antibody containing the exogenous proteolytic cleavage site binds an epitope on the target cell, that is, the cell that makes up the desired cell population. The recombinant antibody is also referred to herein as the anti-target epitope antibody since it binds an epitope on the target cell.

In one aspect of the methods, a target cell attached to a capture antibody-coated solid surface is detached from the surface by contacting the target cell/surface complex with a protease, where the protease is specific for an exogenous proteolytic cleavage site in the capture antibody. Contact with the protease results in cleavage of exogenous the proteolytic site in the antibody and detaching of the target cell from the surface.

In some embodiments, target cells are isolated from a mixed cell population through use of a recombinant antibody that specifically binds an epitope on the surface of the target cell but not on the surface of non-target cells in the population. Before, during, or after the binding of the target cell epitope by the antibody, the antibody is attached to a surface to enable the separation of the bound target cells from the unbound non-target cells. The recombinant antibody comprises an inserted exogenous proteolytic cleavage site that allows for release or detachment of the cell from the surface through treatment of the cell/antibody/surface complex with a protease specific for the exogenous proteolytic cleavage site. The methods allow for enrichment of target cells or depletion of target cells from a mixed cell population.

In some embodiments of the methods and compositions, the contacting with the protease results in at least 50% of the bound target cells detaching from the surface. In other embodiments, the contacting with the protease results in >60%, >70%, >80%, >90%, or >95% of the bound target cells detaching from the surface.

For example, a method for isolating a target cell from a mixed cell population comprises: a) contacting a mixed cell population with an antibody attached to a solid surface, wherein the antibody specifically binds to the target cell in the population and wherein the antibody comprises an exogenous proteolytic cleavage site; b) separating the target cells bound to the surface from cells not bound to the surface; c) contacting the target cells bound to the surface with a protease, or portion of a protease, specific for the cleavage site to detach the target cells from the surface; and d) collecting the target cells detached from the surface.

In some embodiments, a mixed cell population is incubated with the recombinant anti-target epitope antibody prior to the antibody attaching to a surface. Following the recombinant antibody binding the target cell, the antibody is attached either directly or indirectly to a surface to enable the separation of the bound target cells from the unbound non-target cells. Antibody attachment to the surface following antibody-target cell binding is useful, in particular, for the isolation of a rare cell population, such as, for example, a CD34+ cell population.

Also provided are methods for producing a population of activated target cells, such as, for example, CD3+ T cells which can be activated by incubation with an anti-CD3+ antibody in the presence of IL-2. An example of a method for producing a population of activated target cells comprises: a) contacting a cell population comprising target cells with an agonistic antibody attached to a solid surface, the agonistic antibody specifically binds to the target cells and the antibody comprises an exogenous proteolytic cleavage site; and b) incubating the cells and the antibody attached to the surface under conditions to allow activation of the target cells. The method may optionally further comprise: c) separating the target cells bound to the surface from cells not bound to the surface; d) contacting the target cells bound to the surface with a protease, or portion of a protease, specific for the cleavage site to detach the target cells from the surface; and e) collecting the activated target cells detached from the surface. Such isolated activated cells may be used immediately in particular application or may be expanded prior to use in an application. For example, isolated activated T cells may be expanded 10-fold to 1000-fold in culture prior to use in an in vivo application.

In some embodiments, the exogenous proteolytic cleavage site is inserted into the hinge region of the recombinant antibody. With the exogenous cleavage site in the hinge region, treatment with the specific protease results in cleavage of the recombinant antibody into 3 fragments: 2 Fab fragments and 1 Fc fragment.

The Fc portion of an antibody harbors effector functions and mediates different physiological effects of the immune system in part through binding Fc receptors and other molecules of the immune system. Detaching the target cells from the antibody/surface complex through an exogenous cleavage site which removes the Fc portion of the recombinant antibody from the target cell produces an isolated target cell population free of the Fc domain and its associated effector functions. Accordingly, isolation of target cells using the methods and recombinant antibody provided herein can produce a target cell population free of anti-target epitope antibody Fc domain. For use in cell therapy applications, purification of cells without biologically active antibody (i.e., without the Fc domain) attached is important for the safety and efficacy of the treatments.

The Fab fragments of an antibody are monovalent and bind to the target epitope with lower avidity than the complete antibody, which is at least divalent, binds to the epitope. Thus, the Fab fragments bound to the target epitope on the cell following protease treatment detach from cells more easily than complete antibody. Accordingly, isolation of target cells using the methods and recombinant antibody provided herein can produce an antibody-free target cell population.

In some embodiments, provided is a method for positively isolating an antibody-free population of target cells which comprises: a) contacting a mixed cell population with an antibody attached to a solid surface, the antibody specifically binds to the target cell and the antibody comprises an exogenous proteolytic cleavage site; b) separating the target cells bound to the surface from cells not bound to the surface; c) contacting the surface bound target cells with a protease, or portion of a protease, specific for the exogenous cleavage site in the antibody to detach the target cells from the surface; and d) collecting the target cells detached from the antibody and the surface to form a population of target cells free of bound antibody.

In some embodiments, provided is a method for producing a bead-free population of target cells which comprises: a) contacting a mixed cell population with an antibody attached to a bead, the antibody specifically binds to the target cell and the antibody comprises an exogenous proteolytic cleavage site; b) separating the target cell bound to the bead from cells not bound to the beads; c) contacting the bead bound target cell with a protease, or portion of a protease, specific for the cleavage site to detach the target cell from the bead; and d) collecting the target cells detached from the beads to form a bead-free population of target cells.

The methods and compositions provided herein are for use in isolating or separating any type of cell from others in a mixed cell population so long as the target cell has an epitope that can be distinguished from the other cells in the population. In other embodiments, the methods and compositions are for use in isolating or separating any type of cell from non-cell components of a composition so long as the target cell has an epitope that can be distinguished from the unwanted components of the composition.

In some embodiments of the methods, at least about 15% of the target cells in the starting composition are recovered. In some embodiments, at least about 20%, at least about 25%, at least about 30%, at least about 35%, at least about 40%, or at least about 50% of the target cells are recovered. In certain embodiments, about 15%, 20%, 25%, 30%, 35%, 40%, 45%, 50%, 60%, 70%, 80%, or 90% of the target cells in the starting composition are recovered.

In some embodiments, viability of the isolated target cell population is greater than 50%. In other embodiments, viability of the isolated target cell population is greater than 60%, greater than 70%, greater than 80%, greater than 90%, or greater than 95%. In certain embodiments, viability of the isolated target cell population is about 50%, 60%, 70%, 80%, 90%, or 95%.

As described, the methods and compositions provided can be used for the enrichment of target cells in an isolated cell population. Accordingly, in some embodiments, purity of the isolated target cell population is greater than 50%, that is, the percentage of the total isolated cell population which are target cells is greater than 50%. In other embodiments, purity of the isolated target cell population is greater than 60%, greater than 70%, greater than 80%, greater than 90%, or greater than 95%. In certain embodiments, purity of the isolated target cell population is about 50%, 60%, 70%, 80%, 90%, or 95%.

To capture the target cell, a mixed cell population is incubated with the recombinant anti-target epitope antibody before, during, or after attachment of the recombinant antibody to the surface. In some embodiments, this incubation is performed with the cell and antibody suspension being agitated, for example without limitation, with a rolling motion, a rocking motion, or a stirring motion. In other embodiments, this incubation of the cell and antibody suspension is performed without agitation. This incubation occurs at a temperature and for a length of time suitable for the antibody to bind the epitope on the target cell. In some embodiments, the incubation occurs at room temperature. In other embodiments, the incubation occurs at 4° C., 8° C., 25° C., 30° C., or 37° C. In some embodiments, the incubation is for about 5 minutes to about 2 hours. In certain embodiments, without limitation, the incubation is for about 5 minutes, 10 minutes, 15 minutes, 20 minutes, 30 minutes, 40 minutes, 50 minutes, 60 minutes, 75 minutes, 90 minutes, or 105 minutes.

In provided methods, a target cell/recombinant antibody/surface complex is formed. The components of this complex can be brought together in several different ways. For example, in one embodiment, the composition comprising the target cell can be contacted with the anti-target epitope recombinant antibody attached to the surface. In another embodiment, the composition comprising the target cell can be contacted with the anti-target epitope recombinant antibody and after the target cell has bound the antibody, the antibody can be attached to the surface through, for example, another antibody which links the recombinant antibody to the surface. In yet another embodiment, the composition comprising the target cell can be simultaneously contacted with the anti-target epitope recombinant antibody and with the surface, the recombinant antibody having some means to link the recombinant antibody to the surface.

Thus, in another aspect, provided herein is a composition comprising a target cell, a recombinant antibody, and a solid surface, where the recombinant antibody is specifically bound to the cell and is also attached to the solid surface, and where the recombinant antibody comprises an exogenous proteolytic cleavage site.

The recombinant antibody can be directly attached to the surface or indirectly attached to the surface, for example, through the use of a linker, such as an antibody, which binds the recombinant antibody and which is attached to the surface.

In certain embodiments, the surface is a solid surface. Without limitation, the solid surface can be in the form of a cell culture well or culture plate, a particle or bead, a microscope slide, or a membrane. The solid surface can be magnetic or non-magnetic. In embodiments in which the surface is non-magnetic, separation of the target cells bound to the recombinant antibody/surface complex from non-target cells and material can be accomplished, for example, through washing, centrifugation, filtration and/or sieving. Non-magnetic particles suitable for use in the provided methods, such as polymer beads, are commercially available and techniques for attaching an antibody to such beads are well known in the art.

The use of magnetism in cell isolation techniques is well known and magnetic surfaces for use in such techniques are commercially available. Magnetic or magnetizable surfaces, such as particles, for use in the methods can be without limitation, magnetic, paramagnetic, or superparamagnetic. The term “magnetic” as used herein means that the support is capable of having a magnetic moment imparted to it when placed in a magnetic field, and thus is displaceable under the action of that field. Accordingly, magnetic particles may readily be removed from other components of a sample by magnetic aggregation, which provides a quick, simple, and efficient way of separating the particles following binding of the target cell and also following proteolytic release of the target cell from the antibody/particle complex. In addition, magnetic aggregation is a gentler separation method than techniques which generate shear forces, such as centrifugation.

Thus, magnetic particles may be removed or separated by application of a magnetic field, for example by using a permanent magnet. It is typically sufficient to apply a magnet to the side of the vessel containing the sample mixture to aggregate the particles to the wall of the vessel and to remove the remainder of the sample (e.g., the unbound, non-target cells) and/or the particles (e.g., following protease treatment). Alternatively, separation of the magnetic particles may be performed using an automated system for handling of such magnetic particles. Such automated systems and apparatuses are known and commercially available.

Application of a magnet can be for a time sufficient to segregate the magnetic particles from the remainder of the sample and will depend, for example, on the type of magnetic particle and the composition of the sample. Typically, paramagnetic or superparamagnetic particles of the micron and submicron sizes can be segregated to the side of a vessel containing an aqueous sample through application of a magnet for minutes, for example, 1 to 30 minutes. For example, particle segregation can occur through application of a magnet for 20 minutes or less. In some embodiments using magnetic particles as the as the surface to which the recombinant antibody is attached, segregation of the particles is accomplished through application of a magnet for 1 to 20 minutes. In some embodiments, application of a magnet is for 1, 2, 3, 4, 5, 6, 7, 8, 9, 10, 15, 20, or 30 minutes.

In some embodiments, magnetic particles for use in the cell separation methods are about 1-10 micrometers in diameter. In certain embodiments, the particle diameter is about 1 μm, 2 μm, 2.8 μm, 3.2 μm, 3.5 μm, 4.5 μm, 5 μm, 8 μm, or 10 μm. In other embodiments, the magnetic particles are less than 1 μm in diameter, for example, about 0.05 to 1 μm or about 0.09 to 0.6 μm. In certain embodiments, the particle diameter is about 50 nm, 90 nm, 135 nm, 175 nm, 200 nm, 250 nm, 300 nm, 400 nm, 500 nm, 600 nm, 700 nm, or 800 nm.

Magnetic particles suitable for use in the methods provided are of a variety of compositions including, but not limited to, fine grains of iron oxides dispersed throughout the interior of a polymer particle, silanized particles of magnetic iron oxides, and silanized particles of magnetic porous glass. Polymer compositions of magnetic polymer particles include, for example, polystyrene, cellulose, and latex. Commercially available magnetic particles are available with surfaces modified so as to allow for direct or indirect attachment of molecules, such as a capture antibody, to the particle surface. Such surface modifications of such particles include, but at not limited to, immobilized antibodies such as, without limitation, secondary antibodies, immobilized protein A, immobilized protein G, immobilized streptavidin, immobilized avidin, immobilized biotin, immobilized oligo (dT), and having end groups such as, without limitation, —COOH, —NH2, —OH, epoxy-, tosyl-activated, hydrazide, and glyceryl.

In some embodiments, the target cells are animal cells, such as mammalian cells, including but not limited to, human, non-human primate, murine, porcine, bovine, ovine, feline, canine, and rabbit cells. Target cells include, without limitation, blood cells, thymocytes, lymphocytes, bone marrow cells, splenocytes, umbilical cord blood cells, liver cells, corneal cells, epithelieal cells, endothelial cells, bone cells, adult stem cells, induced pluripotent cells, embryonic stem cells, and mesenchymal stem cells.

In certain embodiments, the target cells are T cells, including without limitation, helper T cells, cytotoxic T cells, memory T cells, regulatory T cells, natural killer T cells. In some embodiments, the target cells are CD3+ T cells. In some embodiments, the target cells are CD4+ T cells. In other embodiments, the target cells are CD8+ T cells. In still other embodiments, the target cells are CD 34+ cells or Tra-1-60+cells.

In some embodiments, the mixed cell population from which the target cell is to be isolated is, without limitation, blood cells, such as peripheral blood mononuclear cells, liver cells, bone marrow cells, umbilical cord blood cells, splenocytes, endothelial cells, and epithelial cells.

In certain embodiments, the methods and compositions provided are for use in isolating a target cell based on an endogenously expressed epitope on the cell. In such methods, the recombinant antibody specifically binds an epitope endogenously expressed on the target cell.

In certain embodiments, the methods and compositions provided herein are for use in isolating a target cell which has been transfected or transduced to express an exogenous receptor or cell surface marker. In such methods, the recombinant antibody specifically binds an epitope of the exogenous receptor or cell surface marker on the target cell.

Target cells and the mixed cell populations may be primary cells, including without limitation, freshly isolated cells, cryopreserved cells, or cultured primary cells, or the cells may be immortalized cells, such as an immortalized cell line. In some embodiments, the primary or immortalized cell may be a transfected or transduced cell or cell line.

In one aspect, a recombinant antibody is provided which comprises an exogenous proteolytic cleavage site for a protease. The exogenous proteolytic site is a particular amino acid sequence which has been added to the antibody amino acid sequence and which can be cleaved by a particular protease. In some embodiments, the exogenous proteolytic cleavage site is inserted in the hinge region of the antibody. For example, the exogeneous cleavage site may be inserted in the upper hinge region, e.g., in the hinge region immediately following the CH1 domain, or may be inserted in the lower hinge region, e.g., in the hinge region immediately preceeding the CH2 domain.

The provided methods use a protease that specifically cleaves the exogenous proteolytic cleavage site inserted in the anti-target epitope antibody. In some embodiments, the protease is an endoprotease including, but not limited to, enteropeptidase, thrombin and Factor Xa. In some embodiments, the protease is not expressed in the target cell. In certain embodiments, the protease is a viral protease including, without limitation tobacco etch virus (TEV) protease, a rhinovirus 3C protease, a tobacco vein mottling virus (TVMV) protease, a plum pox virus protease, and a turnip mosaic virus protease. Other proteases with specific cleavage sites may also be used, as will be clear to the skilled artisan.

It is well known that proteases have catalytic domains and these can be used in place of full length proteases. Accordingly, the methods encompass the use of a full length protease, or an active portion thereof. It is also known that proteases may be modified to alter activity in useful ways, for example, increase activity, improve solubility, and/or decrease autolysis, and encompass the use of such modified proteases, or active portions thereof. Accordingly, in some embodiments, the protease is a TEV protease with an S219V modification, including the modified TEV protease AcTEV™ Protease (Invitrogen™, Life Technologies).

The reaction conditions for optimal antibody cleavage by the protease can vary depending, for example, on the cell type and the protease involved. For example, cleavage of the recombinant antibody by the protease may be improved by the presence of a reducing agent in the cleavage reaction solution. Accordingly, in some embodiments, a reducing agent is present when the target cell attached to the antibody-coated surface is contacted with the protease. In some embodiments, the presence of a reducing agent during the protease cleavage reaction increases the yield of detached target cells by greater than 50% as compared to the reaction in the absence of the reducing agent. In certain embodiments, the presence of a reducing agent during the protease cleavage reaction increases the yield of detached target cells by greater than 60%, greater than 70%, greater than 80%, or greater than 90% as compared to the reaction in the absence of the reducing agent.

Typically, a reducing agent that supports sufficient protease activity to cleave the antibody without significantly decreasing target cell viability is suitable for the methods provided. In some embodiments, a reducing agent is present during the protease cleavage reaction at a concentration which does not reduce cell viability by more than 50%. In certain embodiments, a reducing agent is present during the protease cleavage reaction at a concentration which does not reduce cell viability by more than 40%, more than 30%, more than 20%, or more than 10%. In other embodiments, a reducing agent is present during the protease cleavage reaction at a concentration that results in an isolated target cell population with greater than 50% cell viability. In certain embodiments, a reducing agent is present during the protease cleavage reaction at a concentration that results in an isolated target cell population with greater than 60%, more than 70%, more than 80%, or more than 90% cell viability.

Exemplary reducing agents include, but are not limited to TCEP (tris(2-carboxyethyl)phosphine), DTT (dithiothreitol), NAC (N-acetyl cholate), GSH (glutathione), thioglycolate and 2-ME (beta-mercaptoethanol). Such reducing agents may be used at concentrations including, but not limited to, 0.04 mM to 20 mM. In some instances, a reducing agent may be used at concentrations including, without limitation, 0.04 mM to 1 mM, 0.2 mM to 5 mM, 0.4 mM to 10 mM, or 0.8 mM to 20 mM. Exemplary temperatures for the protease reaction include, but are not limited to, 4oC, room temperature and 37oC. Exemplary incubation times for the protease cleavage step include, but are not limited to 15 minutes to 16 hours, such as 0.5 hour, 1 hour, 2 hours, 4 hours, 6 hours, 8 hours, 12 hours, or 16 hours. In some embodiments, the solution comprising the target cell/antibody/surface complex and protease is agitated during the protease cleavage reaction, for example without limitation, with a rolling motion, a rocking motion, or a stirring motion. In other embodiments, this reaction is performed without agitation.

In general, proteases that cleave a protein at a particular cleavage site are suitable for use in the provided methods. The particular proteolytic cleavage site is inserted into the recombinant antibody amino acid sequence through addition, deletion, and/or modification of amino acids to generate the exogenous proteolytic cleavage site. For many proteases, the amino acid requirements for the proteolytic cleavage sites are known. Exemplary proteolytic cleavage sites of the exemplary proteases for use in the methods include, but are not limited to, DDDDK (SEQ ID NO:1) for enteropeptidase (with cleavage occurring following the Lys), LVPRGS (SEQ ID NO:2) for thrombin (with cleavage occurring between the Arg and Gly), LVPRGS (SEQ ID NO:2) for Factor Xa (with cleavage occurring between the Arg and Gly), ENLYFQG (SEQ ID NO:3) for TEV protease (with cleavage occurring between the Glu and Gly), LEVLFQGP (SEQ ID NO:4) for rhinovirus 3C protease (with cleavage occurring between the Glu and Gly). Some amino acid variation at particular positions in the cleavage site sequence may be compatible with proteolytic cleavage by the protease while at other positions in the sequence, no variation is permitted. For example, a consensus TEV protease cleavage site has been defined as Glu-Xaa-Xaa-Tyr-Xaa-Gln-(Ser/Gly) (SEQ ID NO:5), with Xaa being any amino acid and with cleavage occurring between the Gln and Ser/Gly.

To generate the recombinant antibody, an exogenous proteolytic cleavage site is inserted at a position in a constant region or hinge region of the antibody through mutation or modification of amino acids in the antibody. Modification of the amino acid sequence of an antibody can be accomplished using methods well known in the art and commercially available reagents and kits.

The recombinant antibody may be generated using any antibody amino acid or nucleic acid sequence including, for example, mouse, human, rabbit, rat, chicken, goat, Camelid mammal (for example, llama, camel, and alpaca), and cartilaginous fish (for example, shark) antibody sequences. In some embodiments, the recombinant antibody may comprise portions or regions from more than one original antibody or source. For example, a recombinant antibody may contain constant regions and hinge region of a mouse antibody and variable regions of a rabbit antibody. Alternatively, a recombinant antibody may be entirely based on a mouse antibody sequences but the constant regions and variable region sequences may be from two different mouse antibodies. The recombinant antibody may comprise heavy chains from any of IgG, IgM, IgE, IgD, IgA, or IgY, or combinations thereof. The recombinant antibody may comprise either kappa or lamba light chains, or combinations thereof. The recombinant antibody may comprise only heavy chains and lack light chains, such as those found in Camelid mammals and cartilagenous fishes.

As described herein, the recombinant antibody may be a whole antibody molecule, a single chain antibody, or an antibody fragment or portion which retains the ability to exhibit epitope-binding activity. In addition to an epitope-binding portion, the recombinant antibody comprises an exogenous proteolytic cleavage site. Suitable antibody fragments include, but are not limited to, a Fab fragment, a single chain Fv fragment (where both light and heavy chains are present as a fusion protein), and a VHH fragment (a single heavy chain variable domain of an immunoglobulin naturally devoid of light chains, such as can be found, for example, in Camelid mammals and sharks).

In some embodiments, the exogenous proteolytic cleavage site is inserted into a generic constant region backbone of the antibody heavy chain. Variable regions with specificity for any specific cell surface marker can be combined in conjunction with the generic constant region backbone of a heavy chain containing the exogenous cleavage site. Generation of such recombinant antibodies can be accomplished by combining nucleic acid sequences encoding the variable regions of choice with nucleic acid sequences encoding the generic constant regions. Techniques and tools, such as reagents, cells, and devices, to create such recombinant antibodies are well known and commercially available to the skilled artisan.

Also provided are nucleic acid molecules which encode the recombinant antibody. For example, provided is a nucleic acid molecule encoding an antibody heavy chain comprising an exogenous proteolytic cleavage site. In some embodiments, an expression vector comprises the nucleic acid molecule encoding a heavy chain comprising an exogenous proteolytic cleavage site. In some embodiments, such an expression vector comprises a nucleic acid molecule encoding an entire heavy chain including the variable region and constant regions. In other embodiments, such an expression vector comprises a nucleic acid molecule encoding the constant regions of the heavy chain with an insertion site for operatively introducing a nucleic acid molecule encoding a variable region of choice.

The following examples are provided by way of illustration and not by way of limitation.

### EXAMPLES

### Example 1—Generation of Expression Constructs for Encoding Recombinant Antibodies With an Inserted Proteolytic Cleavage Site

For the cloning of the heavy chain constant region (CH) gene of mouse IgG1, the mouse C gamma 1 gene was PCR amplified from genomic DNA isolated from hybridoma cells (BerEp4) using the primers; 5′mIgG1_HindIII gtaagctt tcg ggg aca tgg gaa tgc aaa agt agc (SEQ ID NO:6) and 3′mIgG1_BamHI gtggatcc ggg ctc aaa cca tgg aga ccc ct (SEQ ID NO:7) (restriction enzyme sites HindIII and BamHI are underlined). The blunt PCR product was TOPO cloned into linearized and topoisomerase I-activated pCR®-Blunt II-TOPO vector using Zero Blunt® TOPO® Cloning Kit (Invitrogen™, Life Technologies). A BsmI site in the intron upstream of CH1 of mouse IgG1 was removed by QuikChange® site-directed mutagenesis (Agilent Technologies) using primer 5′ aaGCTTTCGGGGaCaTgGAAATGCAAAAGAGCGGCCttc (SEQ ID NO:8) (mutated BsmI site underlined).

The C gamma 1 gene was modified in the upper hinge region to insert the proteolytic cleavage site of TEV protease using QuikChange primers 5′mIgG1 TEV-Fab:

(SEQ ID NO: 9)
5′-cag Tg ccc agg gat gaa aac ctg tat ttt cag ggc
tgt ggt tgt aag cc

and a complementary 3′ primer. The resulting amino acid sequence in the hinge region was PRDENLYFQGCGCKPCICT (SEQ ID NO:10) (the inserted TEV cleavage site underlined). The TEV cleavage site was introduced upstream of the cysteines in the hinge to enable cleavage into single Fab-fragments when subjected to a TEV protease, such as AcTEV™ protease (Invitrogen™, Life Technologies).

To clone the mouse IgG1-TEV construct into an expression vector with Variable (V-) and Constant (C)-region cloning cassettes, the constructed C gamma 1-TEV fragment was cut from the pCR®-Blunt II-TOPO vector using HindIII and EcoRI restriction enzymes and cloned into the antibody expression vector pLNOH2 (Norderhaug et al. (1997) J. Immunol. Methods 204(1):77-87) on respective restriction sites. pLNOH2 is based on pcDNA3.1, with immunoglobulin leader sequence and restriction sites for cloning any VH-genes and CH-genes. The resulting vector pLNO_mIgG1-TEV contains a V-region cassette (BsiWI/BsmI) for cloning of any VH-region in combination with the mouse C gamma 1-TEV.

For the cloning of the light chain constant region gene of mouse kappa, the mouse C kappa (mCk) was PCR amplified from genomic DNA isolated from hybridoma cells (BerEp4) using the primers 5′ mCk HindIII: 5′-ttat aagctt cttaccttatgtgcttgtg (SEQ ID NO:11) and 3′ mCk BamHI: 5′- atat ggatcc tttgtct cta aca ctc att cc (SEQ ID NO:12) (restriction enzyme sites HindIII and BamHI are underlined). The blunt PCR product was TOPO cloned into linearized and topoisomerase I-activated pCR®-Blunt II-TOPO vector using Zero Blunt® TOPO® Cloning Kit (Invitrogen™, Life Technologies).

To clone the nucleic acid molecule encoding mouse C kappa into an expression vector, the cloned mCk fragment was cut from pCR®-Blunt II-TOPO vector using HindIII and BamHI and cloned into pLNOk. The resulting vector pLNO_mCk contains a VL-region cassette (BsiWI/BsmI) for cloning of any VL-region in combination with the mouse C kappa region.

### Example 2—Cloning Variable Heavy (VH) and Variable Light (VL) Chain Regions Genes from Anti-Human CD3 Expressing Hybridoma Cell Line into Expression Vectors

VH and VL chain genes were PCR amplified from poly dCTP 3′-tailed cDNA. The cDNA was synthesized from total RNA isolated from SPV-3tB hybridoma cells expressing anti-human CD3 antibodies using a gene specific 3′ primer (cDNA_kappa: tgc tgt ctt tgc tgt cct gat (SEQ ID NO:13) and cDNA_IgG2a; tag agg tca gac tgc agg aca (SEQ ID NO:14)). The V-genes were PCR amplified from the poly dCTP-modified cDNA using a 5′ poly G primer and a C-region gene specific 3′ primer (3′ IgkC_rev: caa gaa gca cac gac tga ggc (SEQ ID NO:15), and 3′ IgG2a rev: ctt gac cag gca tcc tag agt ca (SEQ ID NO:16)). The blunt PCR products were TOPO cloned into linearized and topoisomerase I-activated pCR®-Blunt II-TOPO vector using Zero Blunt® TOPO® Cloning Kit, sequenced and analysed for productive V-gene sequence (IMGT database).

New V-region specific primers, with BsmI and BsiWI restriction enzyme sites to enable cloning into the backbone expression vectors pLNO_mIgG1-TEV and pLNO_mCK (Example 1), were synthezized to PCR amplify the cloned VH and VL sequences from the pCR®-Blunt II-TOPO vectors. Primers for anti-human CD3 VL were 5′ VL_BsmI: 5′-attc tgcattc caa att gtt ctc acc cag tct cca gc (SEQ ID NO:17) and 3′ VL_BsiWI: 5′-tcga cgtacg ttct actc acg ttt cag ctc cag ctt ggt cc (SEQ ID NO:18). Primers for anti-human CD3 VH were 5′ VH_BsmI: 5′-tc tgcattc cag gtc cag ctg cag cag (SEQ ID NO:19) and 3′ VH_BsiWI: 5′-ga cgtacg ttct actc acc tga gga gac ggt gac tga cc (SEQ ID NO:20) (restriction enzyme site underlined and splice site in bold). The PCR products encoding VH and VL chain genes were restriction enzyme cut using BsmI and BsIWI and ligated into their corresponding backbone vector described above, resulting in a complete genomic L-chain gene expression vector (pLNO_mCk-SPV) and a complete genomic H-chain gene expression vector (pLNO_mIgG1TEV-SPV) encoding recombinant antibodies with specificity for human CD3 and with the TEV proteolytic cleavage site genetically inserted the upper hinge region.

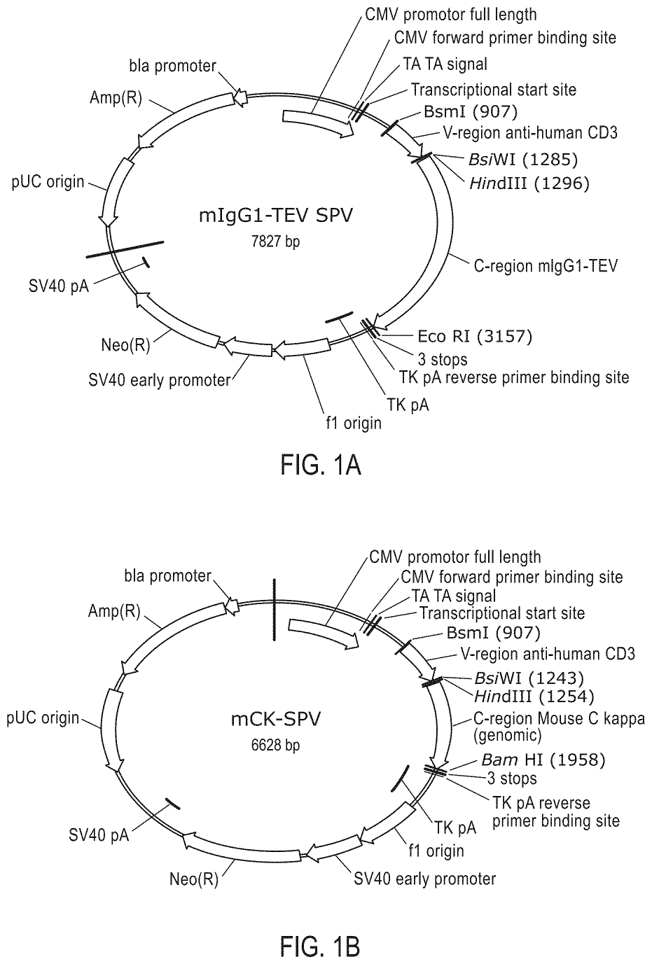

The complete V- and C-region cassettes including the leader region from the vectors pLNO_mCk-SPV and pLNO_mIgG1TEV-SPV were PCR amplified and TOPO cloned into a pcDNA3.3 vector with a mutated BsmI site in the SV40 polyA site. The 5′ primer upstream of the leader sequence was used for both the H-, and L-chain vector (5′ Leader: 5′- cca agctagc ttggtaccg (SEQ ID NO:21)). The 3′ primer was constructed specifically for mCk (3′ mCk: 5′- ggatcctttgtctctaacac (SEQ ID NO:22)) or mIgG1-TEV (3′m IgG1: 5′-gaattcgcccttgtggatc (SEQ ID NO:23)). The PCR products including the leader sequence and Kozak site were amplified using Taq polymerase to ensure TA′ overhangs necessary for TOPO cloning using pcDNA3.3-TOPO® Cloning kit (Invitrogen) and correct orientation of the insert downstream of the CMV promotor was verified. Maps of the H-chain and L-chain expression vectors are depicted in FIG. 1 (mIgG1-TEV SPV (FIG. 1A) and mCk-SPV (FIG. 1B)).

### Example 3—Expression of Recombinant Antibody With an Inserted Proteolytic Cleavage Site

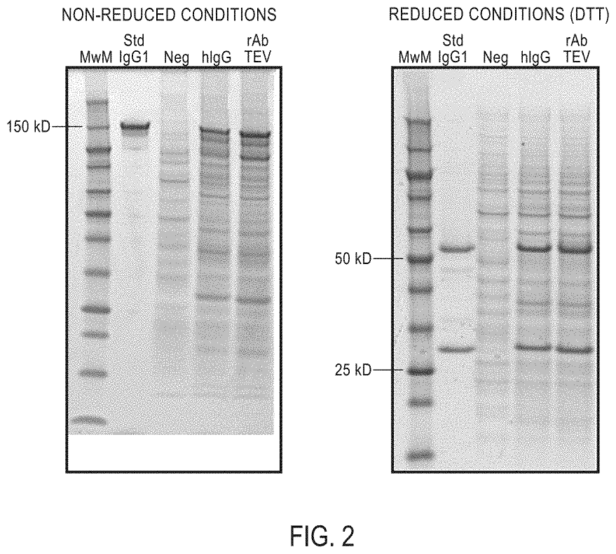

Vectors encoding the anti-CD3 L-chain and H-chain were co-transfected into suspension FreeStyle™ 293-F cells (Invitrogen) using FreeStyle™ 293F Expression System in ratio 1:2 heavy and light chain. The supernant was harvested at day 5 and analysed on a Coomassie gel (NuPAGE® gel, Novex®, Life Technologies) under reduced (with DTT) and non-reduced conditions (FIG. 2). Recombinant antibody expression was estimated to vary between 40-80 μg/ml. The recombinant antibodies were purified from supernantant using Dynabeads® Protein G (Invitrogen™, Life Technologies) and eluted in 50 mM Glycine pH 2.8. The eluate was neutralized to pH 7 by adding Tris-HCl pH 8. The recombinant anti-human CD3 antibody with TEV cleavage site in the upper hinge region of mouse IgG1 is herein referred to as rAb-TEV.

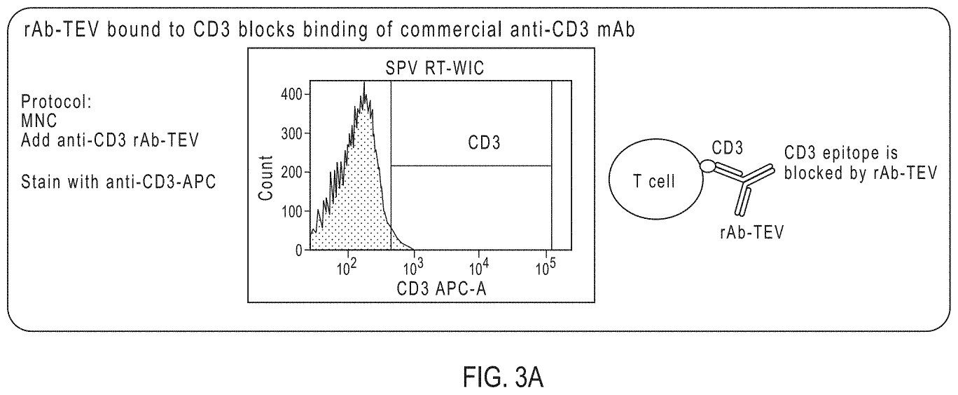

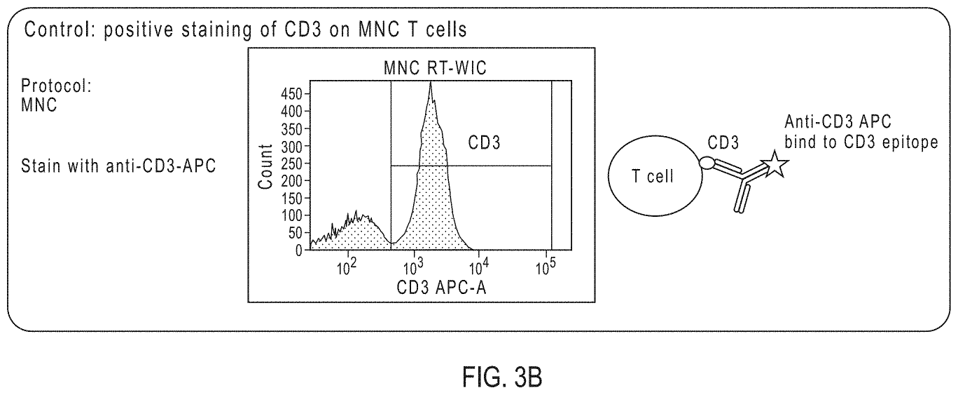

To demonstrate V-region specificity of the constructed rAb-TEV, the purified rAb-TEV was added to human mononuclear cells (MNC) prior to staining with commercially available anti-human CD3 antibody conjugated with APC (anti-CD3-APC). MNC were also stained with anti-CD3-APC alone as a positive control. The cells were then analyzed by flow cytometry. The rAb-TEV blocked staining by the anti-human CD3 antibody, indicating that rAb-TEV binds to the same CD3 epitope (FIG. 3 A-B). This result demonstrates that the cloned VH- and VL-chain regions maintained their specificity for human CD3.

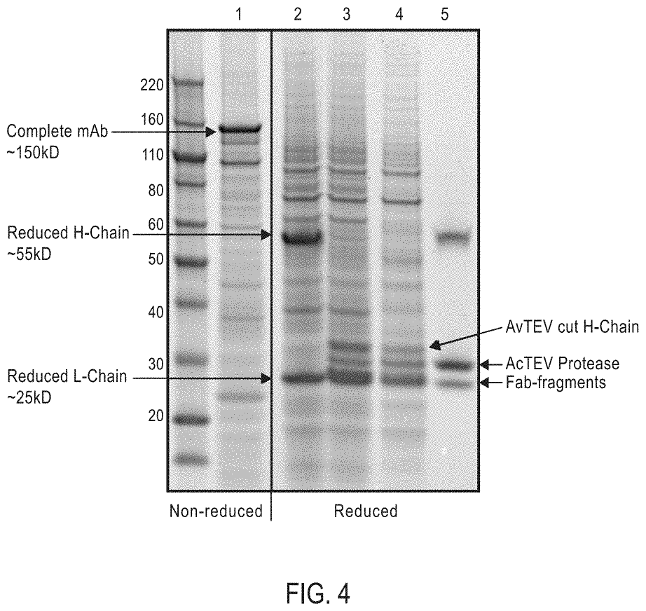

Purified rAb-TEV was incubated with AcTEV™ Protease (Invitrogen™, Life Technologies) in enzyme buffer (1 mM DTT) and the cleaved protein fragments were analyzed on SDS-PAGE Coomassie blue gel under reduced and non-reduced conditions. The results are shown in FIG. 4. When treated with AcTEV™ Protease, the complete IgG1 was cleaved into Fc-fragments and single Fab-fragments demonstrating that the cleavage site is accessible to the protease in the recombinant antibody when inserted in the upper hinge region.

### Example 4—Recombinant Antibody Attached to Solid Surface

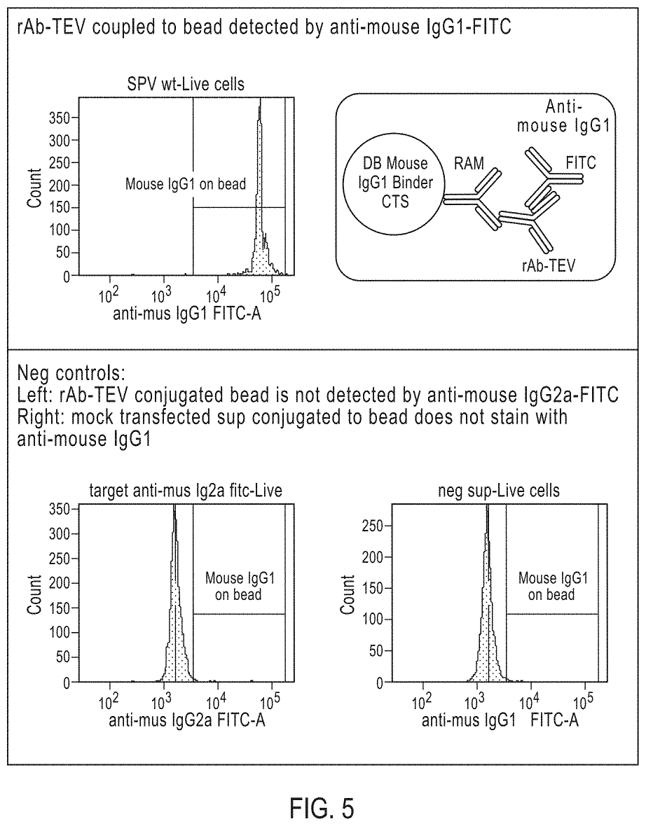

Purified rAb-TEV was coupled to Dynabeads® Mouse IgG1 Binder (Invitrogen™, Life Technologies) which are 4.5 μm paramagnetic particles conjugated with a monoclonal rat-anti mouse IgG1 antibody (RAM). The coupled rAb-TEV of IgG1 isotype could be detected in flow by FITC conjugated anti-mouse IgG1 Ab (FIG. 5, top panel). Negative controls used were FITC conjugated anti-mouse IgG2a (FIG. 5, bottom left) and mock transfected supernanant bound to beads (FIG. 5, bottom right) which were not detected by FITC conjugated anti-mouse IgG1 Ab. These results demonstrate specific binding of mouse IgG1 rAb-TEV to Dynabeads® Mouse IgG1 Binder.

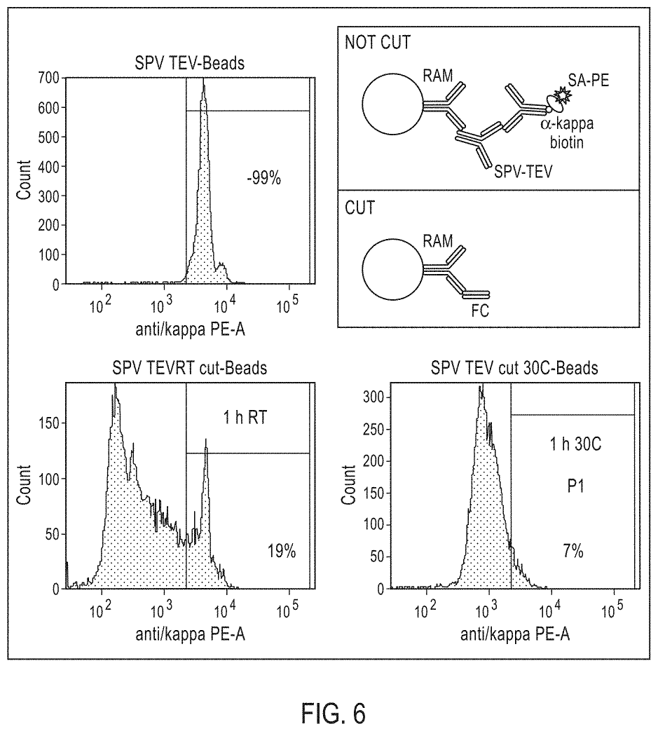

To assess cleavage of rAb-TEV immobilized to a solid surface, rAb-TEV coupled to Dynabeads® Mouse IgG1 Binder beads were incubated with AcTEV™ Protease for 1 hour at room temperature or 37° C. Without AcTEV™ Protease, the beads stained brightly by a PE-conjugated anti-kappa Ab in flow cytometry analysis with 99% of the beads kappa-positive (FIG. 6, top left). However, after a one hour incubation with the protease, the kappa-positive faction of beads was greatly reduced to 19% for room temperature incubation (FIG. 6, bottom left) and to 7% for 37° C. incubation (FIG. 6, bottom right), indicating that the Fab-fragments had been cleaved from the Fc-region of rAb-TEV/bead complex. A cartoon depicting the two scenarios is shown in FIG. 6, top right. Accordingly, the inserted exogenous proteolytic cleavage site was accessible to the protease when the recombinant antibody was attached to the solid surface.

### Example 5—Depletion of Target Cells From Mixed Population Using Recombinant Antibody Attached to Solid Surface

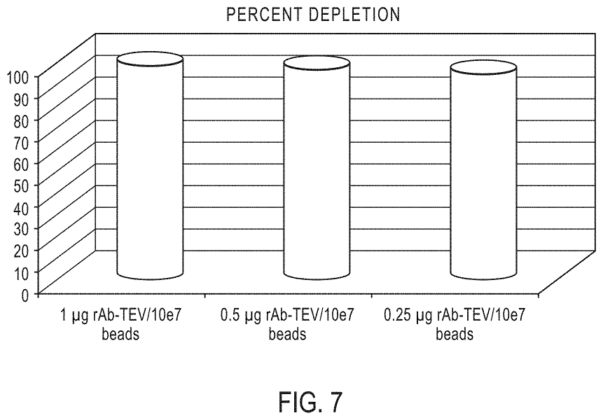

The rAb-TEV was coupled to Dynabeads® Mouse IgG Binder at different concentrations and used to deplete CD3⁺T cells from a mixed population of mononuclear cells (MNC). Four beads per CD3⁺target cell were added and percentage of depleted CD3⁺T cell was analysed by flow cytometry after 20 minutes incubation. All beads efficiently depleted CD3+ T cells in the MNC population with all of the 3 different rAb-TEV/Dynabeads tested binding and depleting more than 94% of CD3⁺T cells (range 94-98%, FIG. 7). The depletion efficiency is similar to Dynabeads CD3 which is the corresponding 4.5 μm magnetic particle covalently conjugated with the SPV-3tB anti-human CD3 Ab (the parent monoclonal Ab of rAb-TEV from where the anti-human CD3 V-regions were cloned), indicating that the V-region genes cloned into rAb-TEV maintained their specificity and functionality when bound to Dynabeads® Mouse IgG1 Binder. The depletion was robust in the range tested (0.25 to 1 μg rAb-TEV per 10E7 beads).

### Example 6—Isolation of Bead-Free CD3+ T Cells Using Recombinant Antibody Attached to Beads Plus Protease

To obtain cell release after bead capture, the protease must have access to the proteolytic cleavage site which could be hidden in the close bead-cell complex. Also, to release a cell from the bead, the majority of epitope bound recombinant antibody (e.g., CD3 bound rAb-TEV) must be cleaved. The density of recombinant antibody coupled to the beads could therefore be a factor in achieving an efficient release and high recovery of isolated cells. Using the anti-CD3 recombinant antibody rAb-TEV and Dynabeads® Mouse IgG Binder, 4 different bead complexes with different amounts of rAb-TEV bound were tested.

Peripheral MNC were isolated from a healthy donor. Four rAb-TEV coupled Dynabeads were added per target CD3+ T cell in the mixed cell sample. The cells were incubated on a roller for 20 min before the non-bead bound cells were removed by placing the tube in a magnet. The washed bead-cell complexes were resuspended in 400 μl release buffer containing 10 U AcTEV™ Protease and incubated on a roller for 2 hours at room temperature. After incubation, the sample was mixed well and placed in the magnet to remove the beads. The supernatant containing the isolated bead-free cells was transferred to a new tube where recovery and purity of the isolated sample was analyzed. A cartoon of the workflow is depicted in FIG. 8.

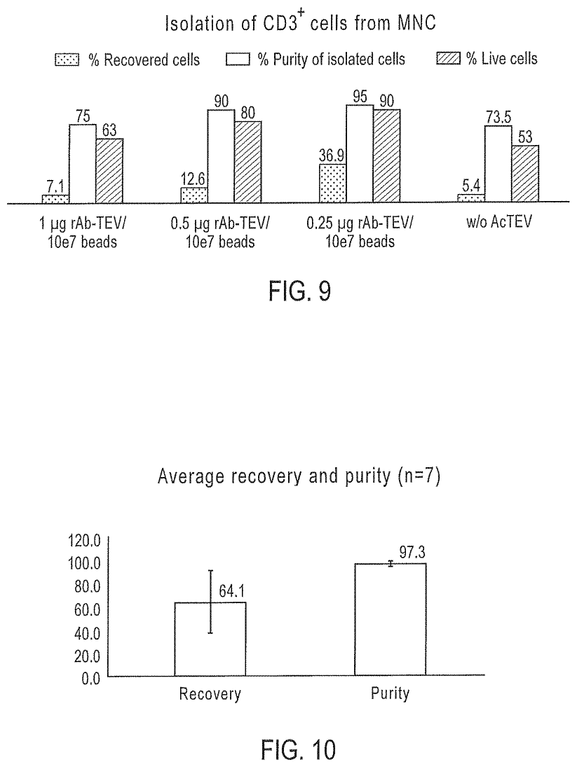

The isolated cell samples were analysed by flow cytometry and the resulting yield, purity and viability are shown in FIG. 9. With a low density of rAb-TEV bound to the beads (0.25 μg per 10E7 beads), 37% of target cells were recovered. With a density of rAb-TEV bound to the beads of 0.5 μg per 10E7 beads, 12.6% of target cells were recovered. In contrast, using 1 μg rAb-TEV bound per 10E7 beads, only 7% of target cells were recovered in the isolated cells sample which was similar to the control where no enzyme was added (5%). In this experiment, the highest recovery of target CD3+ T cells was observed when using the preparation with 0.25 μg rAb-TEV/10E7 Dynabeads Mouse IgG1 Binder. The purity and the viability of these isolated cells were also high (95% and 90%, respectively).

Peripheral MNCs from healthy donors were incubated for 20 min at RT with Dynabeads® Mouse IgG1 Binder coupled with 0.25 μg rAB-TEV per 10E7 beads for cell capture. The cells bound to the rAB were separated from unbound cells using a magnet. The washed bead-cell complexes were resuspended in release buffer containing 10 U AcTEV™ Protease and incubated for 2 hours at room temperature. Beads were removed by magnet and the bead-free, positively isolated CD3+ T cells recovered. After performing this isolation procedure with MNCs from seven healthy donors (n=7), the average recovery of target cells was 64.1% (STDEV 27) and average purity was 97.3% (STDEV 2.2) (FIG. 10). Under these conditions, cell viability was >90%, cell expression of CD2, CD3, CD4, and CD8 was unaffected, and the remaining rAB-TEV Fab fragments were not detected on the isolated cells.

### Example 7—Isolated Cells are Bead-Free and Recombinant Antibody-Free

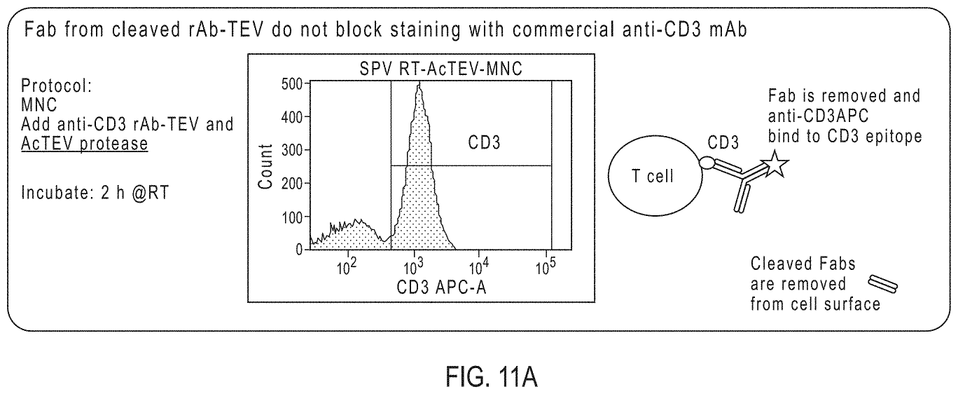

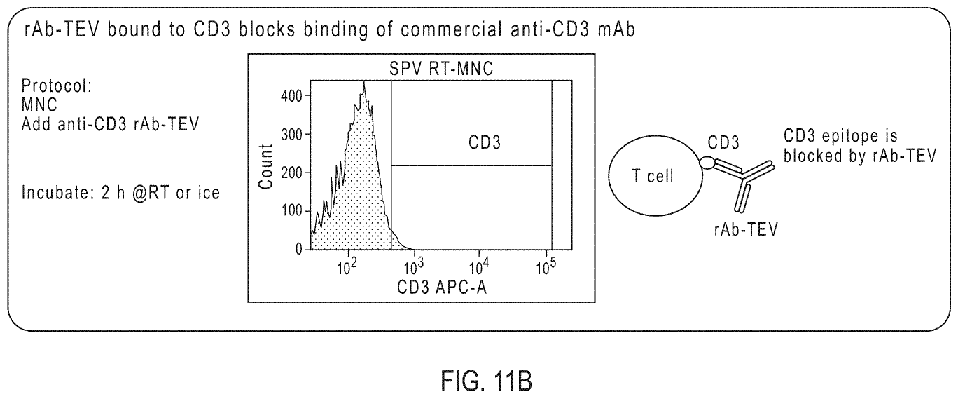

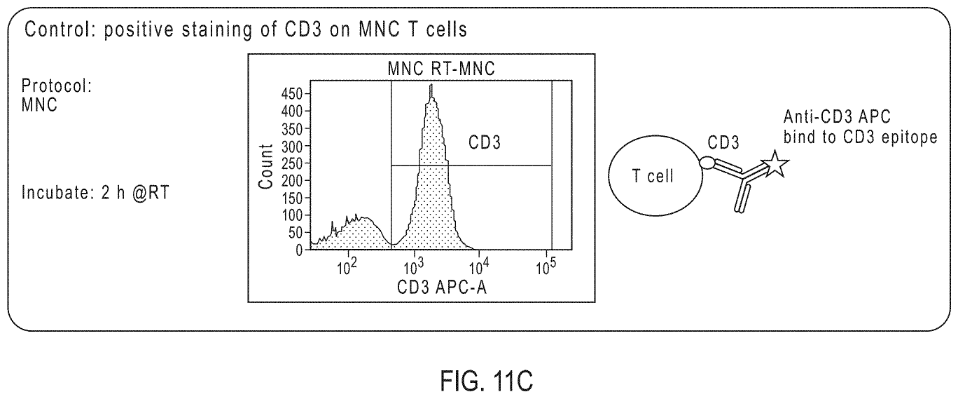

Following cell capture with the recombinant antibody coupled beads, cell release from the beads using AcTEV™ Protease cleaves the CD3 bound rAb-TEV in the upper hinge region resulting in Fab fragments from rAb-TEV that could remain bound to the CD3 epitope on the isolated cells. However, as Fab-fragments have reduced avidity compared to complete IgG antibody, CD3 bound Fab fragments may have higher turn off rate than complete IgG. To assess this, a commercially available anti-human CD3 antibody conjugated with APC (anti-CD3-APC) was used to stain MNC that had been incubated with rAb-TEV with or without the AcTEV™ Protease present. As shown in FIG. 11, the data show that when the protease was present, the CD3 epitope on the cell was no longer blocked by the rAb-TEV and the cells stained with the anti-CD3-APC (FIG. 11A), indicating that Fabs have been effectively removed from the cells after isolation. When the protease was not present, the CD3 epitope on the cell was blocked by the rAb-TEV and the cells did not stain with the anti-CD3-APC (FIG. 11B). Control cells incubated without rAb-TEV and AcTEV™ Protease stained with the anti-CD3-APC (FIG. 11C). Staining the cell populations with labeled anti-kappa antibody support the results found with anti-CD3-APC. These data indicate that the rAb-TEV Fab fragments were effectively removed from the CD3 epitope after cell isolation leaving bead-free and antibody-free cells.

### Example 8—Activation and Expansion of Positively Isolated CD3+ T Cells

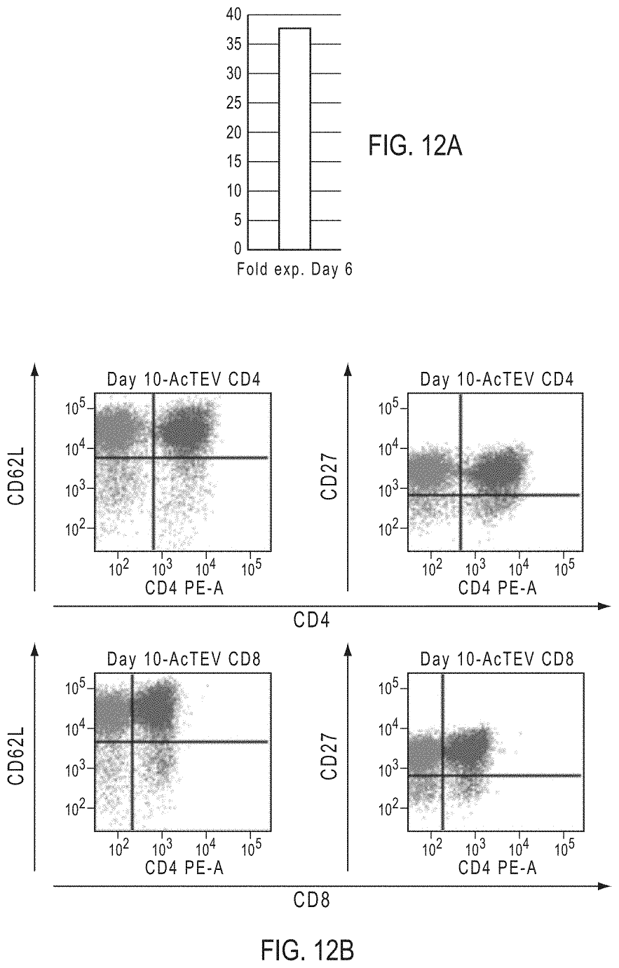

Recombinant antibody rAb-TEV bound to Dynabeads and AcTEV™ Protease were used as described in Example 6 to positively isolate CD3+ T cells from MNCs. These isolated T cells were then activated and expanded in culture using Dynabeads® CD3/CD28 CTS™ according to manufacturer's protocol (Gibco®, Life Technologies). The positively isolated T cells were activated and expanded, showing more than 35 fold expansion by day 6 (FIG. 12A) and a central memory phenotype as shown by CD62L and CD27 expression (FIG. 12B). This amount of activation and expansion is as expected for this type of cell and shows that the isolated cells behave normally. Thus, the cells isolated using the rAb-TEV/bead complex followed by TEV protease treatment were able to be activated and expanded as would be expected for CD3+ T cells isolated from MNC.

### Example 9—Human CD34 Recombinant Antibodies With Protease Cleavage Site

The variable heavy chain region and variable light chain region from the anti-human CD34 hybridoma 561 were cloned into the expression vectors mIgG1-TEV and mCk, respectively, as described above for the anti-CD3 recombinant antibodies. The recombinant antibodies were expressed in FreeStyle™ 293F cells, purified using Protein G, and conjugated to Dynabeads® Mouse IgG1 as described for rAb-TEV with specificity for CD3. See Examples 2-4, for example.

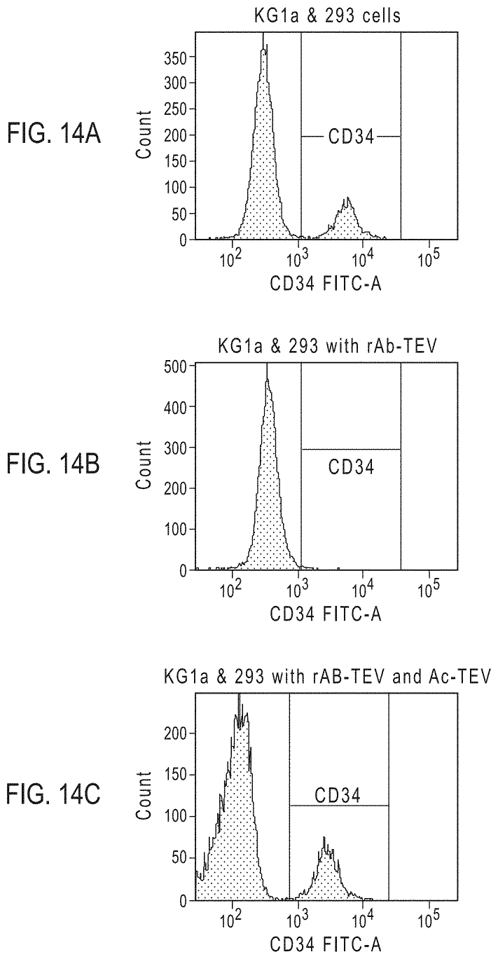

To demonstrate V-region specificity of the constructed rAb-TEV with specificity for CD34 (herein referred to as “rAb-TEV anti-CD34”), the purified rAb-TEV was added to mix of human cells: CD34+ KG1a cells and CD34- FreeStyle™ 293F cells prior to staining with commercially available anti-human CD34 antibody conjugated with FITC (anti-CD34-FITC). The cell mix was also stained with anti-CD34-FITC alone as a positive control. The cells were then analyzed by flow cytometry. The rAb-TEV anti-CD34 blocked staining by the anti-human CD34 antibody, indicating that rAb-TEV binds to the same CD34 epitope (FIG. 14, A and B). This result demonstrates that the cloned VH- and VL-chain regions maintained their specificity for human CD34.

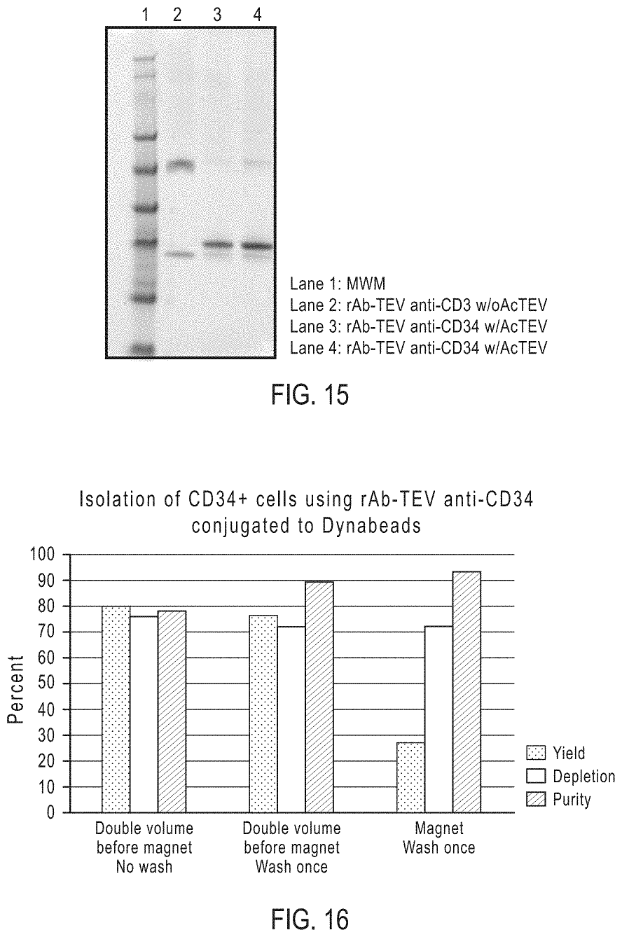

Purified rAb-TEV CD34 was incubated with or without AcTEV™ Protease for 2 hours at room temperature (22° C.) and then analyzed on an SDS-PAGE Coomassie blue gel under reduced conditions. The results are shown in FIG. 15. When treated with the protease, the complete IgG1 was cleaved into Fc-fragments and single Fab-fragments demonstrating that the cleavage site is accessible to the protease in the recombinant antibody.

### Example 10—rAb-TEV Anti-CD34 and Isolation of CD34+ Target Cells

rAb-TEV anti-CD34 conjugated to Dynabeads® mouse IgG1 was used to positively isolate CD34⁺KG1a cells as target cells from a mixed cells population of KG1a cells and FreeStyle™ 293F cells. The density of rAb-TEV anti-CD34 bound to the beads was 0.25 μg per 10E7 beads and four rAb-TEV anti-CD34 coupled Dynabeads were added per target CD34⁺KG1a cell in the mixed cell sample. The cells were incubated on a roller for 20 minutes before the non-bead bound cells were removed by placing the tube in a magnet.

Samples of cells were subjected to varying wash conditions prior to being subject to identical enzymatic cleavage conditions. In one sample (FIG. 16, “double volume before magnet”), a volume of PBS equal to the volume in the tube containing the beads and cells was added to the tube, the tube placed in the magnet, and the non-bead bound cells removed. In another sample (FIG. 16, “double volume before magnet; wash once”), a volume of PBS equal to the volume in the tube containing the beads and cells was added to the tube, the tube placed in the magnet, and the non-bead bound cells removed. An additional volume of PBS wash was added to the bead-bound cells, the tube placed in the magnet, and the non-bead bound cells removed. In another sample (FIG. 16, “magnet; wash once”), the tube containing the beads and cells was placed directly in the magnet (without adding PBS) and the non-bead bound cells removed. A volume of PBS was added to the bead-bound cells, the tube placed in the magnet, and the non-bead bound cells removed.

The washed bead-cell complexes were resuspended in 400 μl per 10 million cells of release buffer containing 10 U AcTEV™ Protease and incubated on a roller for 2 hours at room temperature. After incubation, the sample was mixed well and placed in the magnet to remove the beads. The supernatant containing the isolated bead-free cells was transferred to a new tube and the cell samples were analyzed by flow cytometry for the resultant recovery, depletion, and purity. Results of exemplary CD34+ cell isolations are shown in FIG. 16.

The beads efficiently depleted CD34+ KG1a cells from the mixed population of cells. Target cells were efficiently recovered and the purity of the isolated cells was also high. It was generally found that bead-free CD34+ cells were isolated with comparable purity and recovery as was obtained for the CD3+ T cells described herein.

To assess whether Fab fragments from rAb-TEV anti-CD34 were present on the surface of isolated, bead-free cells following protease treatment, KG1a cells were incubated with rAb-TEV anti-CD34 with or without the AcTEV™ Protease present for 2 hours prior to washings and staining with a commercially available anti-CD34 antibody. Anti-CD34-FITC was then used to stain KG1a cells that had been incubated with rAb-TEV with or without the protease.

As shown in FIG. 14B and FIG. 14C, the when the protease was present, the CD34 epitope on the cell was no longer blocked by the rAb-TEV anti-CD34 and the cells stained with the anti-CD34-FITC (FIG. 14C), indicating that CD34-bound Fab fragments have been effectively removed from the cells by the protease. These data indicate that this isolation method results in bead-free and antibody-free cells.

1. (canceled)

2. A method for isolating a target cell from a mixed cell population, comprising:
a) contacting a mixed cell population with an antibody attached to a solid surface, wherein the antibody specifically binds to the target cell in the population and wherein the antibody comprises an exogenous proteolytic cleavage site.

3. The method of claim 2, further comprising:
b) separating the target cells bound to the surface from cells not bound to the surface;
optionally further comprising c) contacting the target cells bound to the surface with a protease, or portion of a protease, specific for the cleavage site to detach the target cells from the surface; and
optionally further comprising d) collecting the target cells detached from the surface.

4. A method for producing a population of activated target cells, comprising:
a) contacting a cell population comprising target cells with an agonistic antibody attached to a solid surface, wherein the antibody specifically binds to the target cells in the population and wherein the antibody comprises an exogenous proteolytic cleavage site; and
b) incubating the cells and the antibody attached to the surface under conditions to allow activation of the target cells.

5. The method of claim 4, further comprising:
c) separating the target cells bound to the surface from cells not bound to the surface;
optionally further comprising d) contacting the target cells bound to the surface with a protease, or portion of a protease, specific for the cleavage site to detach the target cells from the surface; and
optionally further comprising e) collecting the activated target cells detached from the surface.

6.-7. (canceled)

8. The method of claim 2, wherein the protease is not expressed by the target cell.

9. The method of claim 2, wherein the protease is an endoprotease.

10. The method of claim 2, wherein the protease is a viral protease.

11.-13. (canceled)

14. The method of claim 2, wherein the cleavage site is in the hinge region of the antibody.

15. The method of claim 14, wherein the cleavage site is in the upper hinge region of the antibody.

16. The method of claim 2, wherein the mixed cell population comprises blood cells.

17.-18. (canceled)

19. The method of claim 2, wherein the target cell is a T cell.

20.-22. (canceled)

23. The method of claim 2, wherein the antibody specifically binds to an exogenous protein on the target cell surface.

24. The method of claim 23, wherein the antibody specifically binds a CD3 epitope.

25.-28. (canceled)

29. The method of claim 2, wherein the antibody is indirectly attached to the solid surface.

30. The method of claim 29, wherein the solid surface is a bead.

31.-33. (canceled)

34. A recombinant antibody comprising an exogenous proteolytic cleavage site in the hinge region.

35. The antibody of claim 34, wherein the antibody specifically binds a cell surface epitope.

36.-37. (canceled)
